(19) United States
(12) Patent Application Publication
(10) Pub. No.: US 2024/0342152 A1
(43) Pub. Date: Oct. 17, 2024

(54) Method of Treatment

(71) Applicant: Vanda Pharmaceuticals Inc., Washington, DC (US)

(72) Inventors: Mihael Polymeropoulos, Washington, DC (US); Gunther Birznieks, Washington, DC (US)

(21) Appl. No.: 18/677,361

(22) Filed: May 29, 2024

Related U.S. Application Data
(63) Continuation of application No. PCT/US23/84504, filed on Dec. 18, 2023.
(60) Provisional application No. 63/476,161, filed on Dec. 19, 2022.
(60) Provisional application No. 63/470,574, filed on Jun. 2, 2023.

(51) Int. Cl. A61K 31/454; A61P 25/18; C12Q 1/6876

(52) U.S. Cl. CPC A61K 31/454; A61P 25/18; C12Q 1/6876; C12Q 2600/106

(57) ABSTRACT

Embodiments of the invention relate generally to the treatment of schizophrenia or bipolar I disorder and, more particularly, the administration of iloperidone, including titrated administration of iloperidone, a pharmaceutically-acceptable salt of iloperidone, an active metabolite of iloperidone, or a pharmaceutically-acceptable salt of a metabolite of iloperidone in such treatment. A first aspect of the invention provides a method of treating a patient suffering from bipolar I disorder, the method comprising: administering to the patient an effective amount of iloperidone, a pharmaceutically-acceptable salt of iloperidone, an active metabolite of iloperidone, or a pharmaceutically-acceptable salt of a metabolite of iloperidone, wherein the effective amount is an amount effective to improve at least one symptom of mania in the patient.

### CROSS-REFERENCE TO RELATED APPLICATIONS

This application is a continuation of co-pending PCT Patent Application Serial No. PCT/US23/84504, filed 18 Dec. 2023, which claims priority to then-co-pending U.S. Provisional Patent Application Ser. Nos. 63/476,161, filed 19 Dec. 2022, and 63/470,574, filed 2 Jun. 2023, each of which is hereby incorporated herein as though fully set forth.

### BACKGROUND

### Iloperidone

Iloperidone (FANAPT®) (1-[4-[3-[4-(6-fluoro-1,2-benzisoxazol-3-yl)-1-piperidinyl]propoxy]-3-methoxyphenyl]ethanone), a mixed D₂, 5-HT_{2A0} and α₁-adrenergic antagonist, is an atypical antipsychotic approved by the FDA for the treatment of schizophrenia in adults.

The cytochrome P450 2D6 gene (CYP2D6), located on chromosome 22, encodes the Phase I drug metabolizing enzyme debrisoquine hydroxylase. Many drugs are known to be metabolized by debrisoquine hydroxylase, including iloperidone. Mutations in the CYP2D6 gene have been associated with several drug metabolism-related phenotypes. These include the ultra-rapid metabolizer (UM), extensive metabolizer (EM), intermediate metabolizer (IM), and poor metabolizer (PM) phenotypes.

Where a particular drug is capable of producing unwanted physiological effects in its metabolized or non-metabolized forms, it is desirable to determine whether a patient is a poor metabolizer of the drug prior to its administration. Among the unwanted physiological effects associated with an increased concentration of iloperidone or its metabolites are prolongation of the electrocardiographic QT interval and the inducement of orthostatic hypotension (OH).

Methods of administering iloperidone to a patient based on the patient's CYP2D6 genotype are described, for example, in U.S. Pat. No. 10,272,076, which is hereby incorporated herein as though fully set forth. In general, patients known to be CYP2D6 poor metabolizers are administered a reduced dose (typically one-half) of iloperidone as compared to patients known not to be CYP2D6 poor metabolizers.

Known poor metabolizer genotypes described in the '076 patent include, for example, the AA and AG genotypes at CYP2D6G1846A and the TT and CT genotypes at CYP2D6C100T. While the CYP2D6G1846A (AA or AG) genotypes and the CYP2D6C100T (CT or TT) genotypes are specifically described therein, the method of the invention can employ other genotypes that result in decreased activity of the CYP2D6 protein on iloperidone and/or its metabolites. It is within the skill of the art to identify additional CYP2D6 genotypes that result in decreased enzymatic activity on iloperidone and/or its metabolites.

Similarly, patients being treated with a strong CYP2D6 inhibitor or CYP3A4 inhibitor are administered a reduced dose (typically one-half) of iloperidone as compared to patients not being treated with a strong CYP2D6 inhibitor or CYP3A4 inhibitor.

To minimize OH and/or QT prolongation, iloperidone is typically administered twice per day (b.i.d.) and titrated upward over several days from a lower initial dose to reach a therapeutic or effective dose. The FDA-approved titration is 1 mg twice per day (2 mg/day b.i.d.) on day 1, 2 mg twice per day (4 mg/day b.i.d.) on day 2, 4 mg twice per day (8 mg/day b.i.d.) on day 3, 6 mg twice per day (12 mg/day b.i.d.) on day 4, 8 mg twice per day (16 mg/day b.i.d.) on day 5, 10 mg twice per day (20 mg/day b.i.d.) on day 6, and 12 mg twice per day (24 mg/day b.i.d.) on day 7 and each day thereafter.

A post hoc analysis has demonstrated that when patients receiving iloperidone continue on therapy past the titration period, the efficacy of iloperidone in treating schizophrenia is comparable to that of haloperidol and risperidone. This highlights the need to titrate iloperidone to therapeutic levels as expediently as possible.

### Bipolar I Disorder

Bipolar I disorder (bipolar mania) is a manic-depressive illness characterized by the occurrence of at least one manic episode and may present with or without mixed or psychotic features. Most patients also exhibit one or more depressive episodes and often have had one or more major depressive episodes. Bipolar I disorder may coexist with other disorders, such as post-traumatic stress disorder (PTSD), substance use disorders, and mood disorders. Bipolar disorder is estimated to affect 2.8% of the United States Population.

A core symptom of bipolar I disorder is disturbance in sleep. During manic episodes, this commonly presents as reduced need for sleep, with 69-99% of individuals with bipolar I disorder reporting a lessened need for sleep or difficulties in falling and/or staying asleep.

### SUMMARY

A first aspect of the invention provides a method of treating a patient suffering from bipolar I disorder, the method comprising: administering to the patient an effective amount of iloperidone, a pharmaceutically-acceptable salt of iloperidone, an active metabolite of iloperidone, or a pharmaceutically-acceptable salt of a metabolite of iloperidone, wherein the effective amount is an amount effective to improve at least one symptom of mania in the patient.

A second aspect of the invention provides a method of treating a patient suffering from schizophrenia, the method comprising: administering to the patient an effective amount of iloperidone, a pharmaceutically-acceptable salt of iloperidone, an active metabolite of iloperidone, or a pharmaceutically-acceptable salt of a metabolite of iloperidone, wherein the effective amount is an amount effective to improve at least one symptom of schizophrenia in the patient and is administered according to a regimen of: 1 mg twice per day on day 1, 3 mg twice per day on day 2, 6 mg twice per day on day 3, 9 mg twice per day on day 4, and 12 mg twice per day on day 5 and each day thereafter; or 1 mg twice per day on day 1, 3 mg twice per day on day 2, and 6 mg twice per day on day 3 and each day thereafter.

A third aspect of the invention provides, in a method of administering iloperidone, a pharmaceutically-acceptable salt of iloperidone, an active metabolite of iloperidone, or a pharmaceutically-acceptable salt of a metabolite of iloperidone to a patient, an improvement comprising: administering 1 mg twice per day on day 1, 3 mg twice per day on day 2, 6 mg twice per day on day 3, 9 mg twice per day on day 4, and 12 mg twice per day on day 5 and each day thereafter.

A fourth aspect of the invention provides, in a method of administering iloperidone, a pharmaceutically-acceptable salt of iloperidone, an active metabolite of iloperidone, or a pharmaceutically-acceptable salt of a metabolite of iloperidone to a patient, an improvement comprising: administering 1 mg twice per day on day 1, 3 mg twice per day on day 2, and 6 mg twice per day on day 3 and each day thereafter.

A fifth aspect of the invention provides, in a method of administering iloperidone, a pharmaceutically-acceptable salt of iloperidone, an active metabolite of iloperidone, or a pharmaceutically-acceptable salt of a metabolite of iloperidone to a patient known to be a CYP2D6 poor metabolizer, an improvement comprising: administering 1 mg twice per day on day 1, 3 mg twice per day on day 2, and 6 mg twice per day on day 3 and each day thereafter.

A sixth aspect of the invention provides, in a method of administering iloperidone, a pharmaceutically-acceptable salt of iloperidone, an active metabolite of iloperidone, or a pharmaceutically-acceptable salt of a metabolite of iloperidone to a patient being treated with a strong CYP2D6 inhibitor, an improvement comprising: administering 1 mg twice per day on day 1, 3 mg twice per day on day 2, and 6 mg twice per day on day 3 and each day thereafter.

A seventh aspect of the invention provides, in a method of administering iloperidone, a pharmaceutically-acceptable salt of iloperidone, an active metabolite of iloperidone, or a pharmaceutically-acceptable salt of a metabolite of iloperidone to a patient being treated with a CYP3A4 inhibitor, an improvement comprising: administering 1 mg twice per day on day 1, 3 mg twice per day on day 2, and 6 mg twice per day on day 3 and each day thereafter.

An eighth aspect of the invention provides a method of administering a therapeutically-effective dose of iloperidone, a pharmaceutically-acceptable salt of iloperidone, an active metabolite of iloperidone, or a pharmaceutically-acceptable salt of a metabolite of iloperidone to a patient, the method comprising: administering 1 mg twice per day on day 1, 3 mg twice per day on day 2, and 6 mg twice per day on day 3 and each day thereafter.

A ninth aspect of the invention provides a method of administering a therapeutically-effective dose of iloperidone, a pharmaceutically-acceptable salt of iloperidone, an active metabolite of iloperidone, or a pharmaceutically-acceptable salt of a metabolite of iloperidone to a patient, the method comprising: administering 1 mg twice per day on day 1, 3 mg twice per day on day 2, 6 mg twice per day on day 3, 9 mg twice per day on day 4, and 12 mg twice per day on day 5 and each day thereafter.

A tenth aspect of the invention provides a method of treating a patient suffering from bipolar I disorder, the method comprising: administering to the patient an effective amount of iloperidone, a pharmaceutically-acceptable salt of iloperidone, an active metabolite of iloperidone, or a pharmaceutically-acceptable salt of a metabolite of iloperidone, wherein the effective amount is an amount effective to improve one or more symptom of bipolar I disorder selected from a group consisting of: decreased sleep, a decreased need for sleep, and denial of a need for sleep.

An eleventh aspect of the invention provides a method of treating a patient suffering from bipolar I disorder, the method comprising: determining or having determined, from a biological sample of the patient, that the patient's genotype includes at least one copy of the rs55837573 single nucleotide polymorphism (SNP); and administering to the patient an effective amount of iloperidone, a pharmaceutically-acceptable salt of iloperidone, an active metabolite of iloperidone, or a pharmaceutically-acceptable salt of a metabolite of iloperidone, wherein the effective amount is an amount effective to improve at least one symptom of mania in the patient.

These and other aspects, advantages, and salient features of the invention will become apparent from the following detailed description.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 is a plot of the least squares mean difference (LSMD) change in the Young Mania Rating Scale (YMRS) in iloperidone- and placebo-treated patients.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

Data from recent studies demonstrate the efficacy of iloperidone in the treatment of bipolar I disorder in adults as well as the tolerability of more rapid iloperidone titration regimes.

### Treatment of Bipolar I Disorder

In a Phase III clinical study of iloperidone as a treatment of acute manic and mixed episodes associated with bipolar I disorder, approximately 400 volunteers with a history of bipolar disorder and suffering from a current episode of mania are randomized to receive either iloperidone or placebo in a 1:1 ratio. Treatment efficacy is assessed using the Young Mania Rating Scale (YMRS), a known rating scale of clinical severity in the core symptoms of mania, as well as the Clinician Global Impression of Severity (CGI-S) and the Clinician Global Impression of Change (CGI-C).

YMRS assessments are made at baseline and during days 7, 10, 14, 21, and 28 using the following key:
0=reports no decrease in sleep;
1=sleeping less than normal amount by up to one hour;
2=sleeping less than normal by more than one hour;
3=reports decreased need for sleep; and
4=denies need for sleep.

Patients receive four weeks of double-blind treatment comprising the administration of 24 mg/d iloperidone b.i.d. (12 mg twice daily), 12 mg/d b.i.d. (6 mg twice daily) for poor metabolizers, or placebo. CYP2D6 non-poor metabolizers are titrated to a clinical dose of 24 mg/day (12 mg twice daily) by administering 1 mg twice per day on day 1, 3 mg twice per day on day 2, 6 mg twice per day on day 3, 9 mg twice per day on day 4, and 12 mg twice per day on day 5 and each day thereafter. CYP2D6 poor metabolizers are titrated to 12 mg/day (6 mg twice daily) by administering 1 mg twice per day on day 1, 3 mg twice per day on day 2, and 6 mg twice per day on day 3 and each day thereafter.

Table 1 below provides demographic and baseline characteristics of study participants.

TABLE 1
Demographics and Other Baseline Characteristics, ITT Population

| | Iloperidone | | Placebo | |
|---|---|---|---|---|
| Characteristics | (n = 198) | | (n = 194) | |
| Mean Age (SD), years | 42.9 | (12.8) | 43.5 | (12.8) |
| Sex, n (%) | | | | |
| Male | 113 | (57.1) | 105 | (54.1) |
| Female | 85 | (42.9) | 89 | (45.9) |
| Race, n (%) | | | | |
| American Indian or Alaska Native | 1 | (0.5) | 2 | (1.0) |
| Asian | 0 | | 3 | (1.5) |
| Black or African American | 59 | (29.8) | 55 | (28.4) |
| Native Hawaiian or Other Pacific | 1 | (0.5) | 0 | |
| Islander | 129 | (65.2) | 121 | (62.4) |
| White | 8 | (4.0) | 13 | (6.7) |
| Other | | | | |
| Ethnic group, n (%) | | | | |
| Hispanic or Latino | 35 | (17.7) | 28 | (14.4) |
| Not Hispanic or Latino | 161 | (81.3) | 163 | (84.0) |
| Not Reported | 1 | (0.5) | 3 | (1.5) |
| Unknown | 1 | (0.5) | 0 | |
| Mean Age (SD) when first diagnosed as | 25 | (11.65) | 26.4 | (11.97) |
| having a psychotic disorder, years | | | | |
| Mean Weight (kg), SD | 85.55 | (18.1) | 87.08 | (19.0) |
| DSM-5 Classification of Bipolar I | | | | |
| Disorder, n (%) | | | | |
| Manic type | 154 | (77.8) | 163 | (84.0) |
| Mixed type | 44 | (22.2) | 31 | (16.0) |
| CYP2D6 Phenotype, n (%) | | | | |
| Non-poor metabolizers | 177 | (89.4) | 183 | (94.3) |
| Poor metabolizers | 15 | (7.6) | 11 | (5.7) |
| Mean Baseline YMRS total score | 29.2 | (5.3) | 28.8 | (4.6) |
| (SD) | | | | |
| Mean Baseline CGI-S total score | 4.6 | (0.7) | 4.6 | (0.7) |
| (SD) | | | | |
| Mean Baseline MADRS total score | 10.2 | (3.7) | 9.9 | (4.0) |
| (SD) | | | | |
| Mean Baseline YMRS, Sleep Item | 2.5 | (0.8) | 2.5 | (0.9) |
| score (SD) | | | | |

Patients treated with iloperidone show a larger improvement than placebo-treated patients. YMRS assessment shows a statistically significant benefit in iloperidone-treated patients as early as the end of week 2. The difference is highly statistically significant (p=0.000008) at the end of week 4.

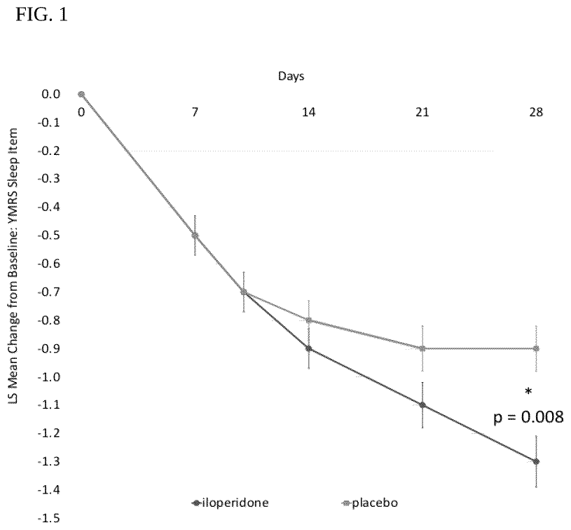

The least squares mean difference (LSMD) in change from baseline to day 28 is statistically significant on the YMRS sleep component in favor of iloperidone versus placebo (iloperidone=−1.26, placebo=−0.95, p=0.008). Results are shown in FIG. 1.

Assessments with the CGI-S and CGI-C also show statistically significant improvement in iloperidone-treated patients as compared to placebo-treated patients (p=0.0005 and 0.0002, respectively).

Table 2 below shows changes from baseline on the YMRS, CGI-S, and CGI-C scales and total MADRS score at endpoint (day 28).

TABLE 2
Change From Baseline (SE) on Efficacy Rating
Scales at Endpoint (Day 28), ITT Population

| | Iloperidone | | Placebo | | | |
|---|---|---|---|---|---|---|
| Parameter | (N = 198) | | (N = 194) | | Diff | p-value |
| YMRS Total | −14.0 | (0.64) | −10.0 | (0.63) | −4.0 | 0.000008 |
| Score***** | | | | | | |
| CGI-S*** | −1.5 | (0.08) | −1.1 | (0.08) | −0.4 | 0.0005 |
| CGI-C*** | 2.3 | (0.09) | 2.8 | (0.09) | −0.5 | 0.0002 |
| MADRS Total | −2.7 | (0.45) | −1.8 | (0.44) | −0.9 | 0.1365 |
| Score | | | | | | |

*p ≤ 0.05;
***p ≤ 0.001;
*****p ≤ 0.00001

Table 3 below shows the frequency of somnolence and sedation as treatment-emergent adverse events.

TABLE 3
Somnolence and Sedation Reported as
Treatment-Emergent Adverse Events

| Parameter, | Iloperidone | Placebo |
|---|---|---|
| n (%) | (N = 206) | (N = 208) |
| Somnolence | 11 (5.3) | 4 (1.9) |
| Sedation | 5 (2.4) | 2 (1.0) |

The incidence of somnolence and sedation is low across treatment groups and likely does not explain YMRS sleep item score improvement.

These results show that iloperidone 24 mg/d (or 12 mg/d for CYP2D6 poor metabolizers) is more effective than placebo in improving both decreased sleep and decreased need or denial of the need for sleep as measured by the sleep component of the YMRS in patients with bipolar I disorder.

Iloperidone also demonstrated statistically significant improvement in the acute treatment of manic or mixed episodes associated with bipolar I disorder in adults as measured by multiple efficacy-related measures, including YMRS total score, CGI-S, and CGI-C, suggesting iloperidone-treatment of mania symptomology may contribute to improved sleep duration and improved perception of need for sleep.

Genetic variants associated with iloperidone response in the treatment of BDI are also observed. Within the iloperidone-treated group (n=167), the variants shown in Table 4 below are associated with a statistically significant change in YMRS.

TABLE 4

| SNP | Gene | YMRS change | p |
|---|---|---|---|
| rs55837573 | RELN | −4.61 ± 0.94 | 2.48 × 10⁻⁶ |
| rs1118464 | NAV2 | −4.15 ± 0.89 | 6.98 × 10⁻⁶ |
| rs76876307 | FAT3 | 15.44 ± 3.28 | 5.27 × 10⁻⁶ |
| rs62300864 | SHROOM3 | 12.08 ± 2.43 | 1.64 × 10⁻⁶ |

The RELN gene codes for an extracellular protein critical for cell positioning and neuronal migration. NAV2 is a neuron navigator gene that may play a role in cell growth and migration. The FAT3 gene codes for an atypical cadherin believed to be involved in cell-cell adhesion and to act upstream of dendrite development and neuron migration. The SHROOM3 gene may play a role in regulating cell shapes. None of these genes is associated with YMRS at baseline, suggesting that the YMRS changes may be attributable to iloperidone treatment.

Drug responders are defined as subjects having an end-of-study YMRS decrease of 50% or more, as compared to baseline. The RELN SNP rs55837573 was identified as the most significantly associated genetic variant among drug responders.

### Tolerability of Faster Iloperidone Titration

A clinical study of adults (n=12, age 18-65, inclusive) with schizophrenia or bipolar I disorder is conducted to assess the tolerability of a faster iloperidone titration scheme. Patients are assigned to one of two cohorts based on CYP2D6 phenotype.

CYP2D6 non-poor metabolizers are titrated to a clinical dose of 24 mg/day (12 mg twice daily) by administering 1 mg twice per day on day 1, 3 mg twice per day on day 2, 6 mg twice per day on day 3, 9 mg twice per day on day 4, and 12 mg twice per day on day 5. CYP2D6 poor metabolizers are titrated to 12 mg/day (6 mg twice daily) by administering 1 mg twice per day on day 1, 3 mg twice per day on day 2, and 6 mg twice per day on day 3. In each case, the target dose of 24 mg/day or 12 mg/day is continued until day 7.

Patients are monitored for OH by measuring blood pressure after lying supine for three minutes and two minutes after standing. OH is defined as a fall in systolic blood pressure of 20 mmHg or more or a fall in diastolic blood pressure of 10 mmHg or more.

The results are compared to historical placebo data from a prior study of the FDA-approved titration regimen.

Only one patient receiving iloperidone met the OH criteria. This one incidence did not have clinical manifestations. This compares to 26 of 147 (17.7%) of placebo-assigned patients in the historical study.

The study results show no other adverse events. There are no deaths, serious treatment emergent events or clinically significant changes in laboratory parameters, ECG findings, or vital signs. And there are no reported incidents of dizziness, syncope, or accidental trauma. Among the placebo-assigned patients in the historical study, 1.4% (n=2) exhibited OH and 6.8% (n=10) reported dizziness.

These results indicate that the faster titration of iloperidone is well-tolerated, with the observed rate of OH similar to historical placebo data. This suggests that it may be possible to reach the therapeutic range (12-24 mg/day administered b.i.d.) of iloperidone one to two days sooner than in the FDA-approved dosing regimen while continuing to mitigate the risk of OH. As noted above, in the treatment of schizophrenia, there is an identified need to titrate iloperidone to therapeutic levels as expediently as possible.

Similarly, the invention described herein permits the treatment of acute episodes of bipolar disorder with accelerated efficacy while still balancing tolerability, particularly with respect to OH and QT prolongation.

Although passages herein refer to administration of “iloperidone,” according to embodiments of the invention, it is understood that a patient in need of treatment may be administered iloperidone, an iloperidone metabolite, or pharmaceutically-acceptable salts thereof. Iloperidone metabolites include the metabolite P88, including its enantiomer forms, S-P88 and R-P88. The S-P88 and/or R-P88 metabolites of iloperidone are described in International Patent Application Publication Nos. WO2003/020707 and WO2013/138602, as well as U.S. Pat. Nos. 7,977,356 and 8,314,129, each of which is incorporated herein by reference as though fully set forth.

The present invention encompasses treatment of a patient for any disease or condition that is ameliorated by administration of iloperidone. As discussed above, such diseases or conditions include, for example, schizoaffective disorders including schizophrenia, depression including bipolar depression, as well as other conditions such as cardiac arrythmias, Tourette's syndrome, psychotic disorders, and delusional disorders.

Iloperidone, iloperidone metabolites, or pharmaceutically-acceptable salts thereof may be administered in any number of manners, as will be recognized by one skilled in the art. Oral administration may be typical, but other routes include, for example, parenteral, nasal, buccal, transdermal, sublingual, intramuscular, intravenous, rectal, vaginal, etc.

According to some embodiments of the invention, iloperidone, an iloperidone metabolite, or a pharmaceutically-acceptable salt thereof may be administered in depot form. Such depot forms are described, for example, in U.S. Pat. Nos. 7,767,230; 8,815,293; 8,293,765; 8,227,488; and 8,614,232, each of which is incorporated herein by reference as though fully set forth. One skilled in the art will understand that, when administered in depot form, the phrase “daily dose” used herein refers to an expected or intended dose to which the individual is effectively exposed during the course of a day.

While this invention has been described in conjunction with the specific embodiments outlined above, it is evident that many alternatives, modifications and variations will be apparent to those skilled in the art. Accordingly, the embodiments of the invention as set forth above are intended to be illustrative, not limiting. Various changes may be made without departing from the spirit and scope of the invention as defined in the following claims.

What is claimed is:

1. A method of treating a patient suffering from bipolar I disorder, the method comprising:
administering to the patient an effective amount of iloperidone, a pharmaceutically-acceptable salt of iloperidone, an active metabolite of iloperidone, or a pharmaceutically-acceptable salt of a metabolite of iloperidone,
wherein the effective amount is an amount effective to improve at least one symptom of mania in the patient.

2. The method of claim 1, wherein improvement of the at least one symptom of mania is measured using one or more of the Young Mania Rating Scale (YMRS), the Clinician Global Impression of Severity (CGI-S), or the Clinician Global Impression of Change (CGI-C).

3. The method of claim 1, wherein the effective amount is between about 8 mg/day and about 24 mg/day.

4. The method of claim 1, wherein the effective amount is between about 8 mg/day and about 16 mg/day.

5. The method of claim 1, wherein the effective amount is between about 12 mg/day and about 24 mg/day.

6. The method of claim 1, wherein the effective amount is administered in a divided dose, twice per day.

7. The method of claim 1, wherein administering includes titrating increasing daily doses of iloperidone up to the effective amount.

8. The method of claim 7, wherein titrating includes administering 1 mg twice per day on day 1, 2 mg twice per day on day 2, 4 mg twice per day on day 3, 6 mg twice per day on day 4, 8 mg twice per day on day 5, 10 mg twice per day on day 6, and 12 mg twice per day on day 7 and each day thereafter.

9. The method of claim 7, wherein titrating includes administering 1 mg twice per day on day 1, 3 mg twice per day on day 2, 6 mg twice per day on day 3, 9 mg twice per day on day 4, and 12 mg twice per day on day 5 and each day thereafter.

10. The method of claim 7, wherein titrating includes administering 1 mg twice per day on day 1, 3 mg twice per day on day 2, and 6 mg twice per day on day 3 and each day thereafter.

11. The method of claim 1, wherein the method further comprises:
determining whether the patient has a CYP2D6 genotype associated with being a CYP2D6 poor metabolizer.

12. The method of claim 11, wherein, in the case that the patient is determined to have a CYP2D6 genotype associated with being a CYP2D6 poor metabolizer, the effective dose is 12 mg/day and administering includes titrating increasing daily doses of iloperidone up to the effective amount according to a regimen of 1 mg twice per day on day 1, 3 mg twice per day on day 2, and 6 mg twice per day on day 3 and each day thereafter.

13. The method of claim 12, wherein the CYP2D6 poor metabolizer genotype is selected from a group consisting of: AA at CYP2D6G1846A, AG at CYP2D6G1846A, TT at CYP2D6C100T, and CT at CYP2D6C100T.
